(19) United States
(12) Patent Application Publication
(10) Pub. No.: US 2004/0014738 A1
(43) Pub. Date: Jan. 22, 2004

(54) Method of inactivating micro-organisms

(76) Inventors: Thomas Martinus Albert Remko Dubbelman, Alphen a/d Rijn (NL); Johannes Wilhelmus Maria Lagerberg, Leiden (NL); Laurence Lilianne Annette Trannoy, Den Haag (NL); Agatha Brand, Amsterdam (NL)

Correspondence Address: PEACOCK MYERS AND ADAMS P C, P O BOX 26927, ALBUQUERQUE, NM, 871256927

(21) Appl. No.: 10/398,923

(22) PCT Filed: Oct. 15, 2001
(86) PCT No.: PCT/NL01/00759

(30) Foreign Application Priority Data
Oct. 13, 2000 (EP) 00203560.8

(51) Int. Cl. A61K 31/555; A61K 31/409

(52) U.S. Cl. 514/185; 514/410

(57) ABSTRACT

The invention relates to a method of inactivating micro-organisms present in a liquid containing stem cells, where the method comprises the steps of -combining said liquid with a photosensitiser, and -activating the photosensitiser. In accordance with the present invention, a positively-charged sensitizer is used having a defined structure. Surprisingly, the use of these compounds minimize the damage to the stem cells.

The present invention relates to a method of inactivating micro-organisms present in a liquid containing stem cells comprising the steps of

combining said liquid with a photosensitiser, and

activating the photosensitiser.

Stem cells are more and more used to grow desired cell types, for example for transplantation purposes and after chemotherapeutic interventions. A major source of stem cells is the umbilical cord. The solution containing the stem cells may be contaminated with various micro-organisms such as viruses (HIV, hepatitis B and C), fungi and bacteria. 10% of the umbillical blood samples appear to be contaminated (unpublished data, submitted for publication). This severely limits or even rules out their use for the desired purpose.

It is known to inactivate organisms present in a blood product, such as plasma, red cells, platelets, leukocytes and bone marrow. For example, U.S. Pat. No. 5,360,734 describes a method comprising the addition of the photosensitiser to the blood product and irradiation with light to activate the photosensitiser, thereby inactivating viral pathogens. Such a method causes damage to the cells, which is, for example, demonstrated by the hemolysis of erythrocytes. The method disclosed in U.S. Pat. No. 5,360,734 is in particular aimed at and reducing the influence of plasma proteins in order to increase the stability of the red cells. Given the intended use of stem cells, damage to the cells should be avoided as much as possible.

The object of the present invention is to provide a method of inactivating micro-organisms in a liquid containing stem cells, minimizing the damage to the stem cells.

Thus, the present invention relates to a method according to the preamble characterized in that as the photosensitiser a compound is used chosen from the group consisting of compounds with the formulas Ia-Id

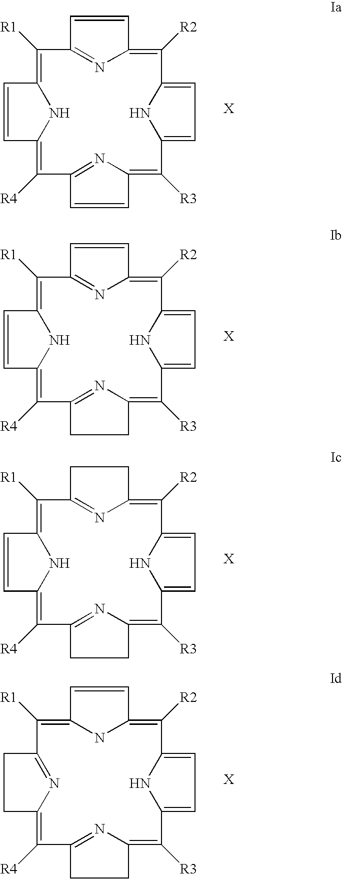

wherein R₁, R₂, R₃ and R₄ are independently chosen from the group consisting of

hydrogen,

a halogen atom,

(C₁-C₂₀)alkyl, (C₁-C₂₀)alkoxy, (C₁-C₂₀)acyl, (C₁-C₂₀)acyloxy, (C₂-C₂₀)alkenyl, or (C₂-C₂₀)alkynyl, each of which may be linear or branched and each of which may be substituted with one or more groups chosen from

hydroxyl,

amino which may be substituted with 1 to 3 groups chosen from (C₁-C₂₀)alkyl, (C₂-C₂₀)alkenyl, (C₁-C₂₀)alkoxy, (C₂-C₂₀)alkynyl, and —(R₅-Z)ₘ—R₆ where R₅ is (CH₂)ₙ, Z is O or S, and R₆ is (C₁-C₂₀)alkyl and m and n are, independently, 1-10, each substituent group of the amino group may be linear or branched and each of these may be substituted with one or more groups chosen from hydroxyl, and a halogen atom,

nitril, and

a halogen atom,

(C₆-C₂₀)aryl, and (C₆-C₂₀) heterocyclic aryl group each of which may be substituted with one or more groups chosen from

hydroxyl,

amino which may be substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which may be substituted with one or more groups chosen from hydroxyl, and a halogen atom,

nitril,

a halogen atom, and

(C₁-C₁₀)alkyl, (C₁-C₁₀)alkoxy, (C₂-C₁₀)alkenyl, the heterocyclic aryl group containing at least one atom chosen from N, O, P, and S where P, N or S may be substituted with a group chosen from (C₁-C₂₀)alkyl, (C₂-C₂₀)alkenyl, (C₁-C₂₀)alkoxy, and (C₂-C₂₀)alkynyl, each of which may be linear or branched and each of which may be substituted with one or more groups chosen from hydroxyl, and a halogen atom,

at least one of the groups R₁, R₂, R₃ and R₄ contains a quaternary nitrogen atom, and wherein X is a pharmaceutically acceptable counterion.

Surprisingly, it has been found that by using a photosensitising compound as disclosed above, the damage to the stem cells is avoided, even if no protecting agent (such as disclosed in PCT/NL99/00387) is present. This is highly surprising, because white blood cells have found to be significantly affected by such a photodynamic treatment. In particular their response to allogenic stimulation is strongly reduced, and also their capability to present antigens is adversely affected. In general, if R₁, R₂, R₃ or R₄ is an aliphatic group, short chains will be preferred, such as those having 1-6 atoms.

In the present invention, when referring to liquid containing stem cells, the term “liquid” is to be understood as any aqueous solution comprising at least stem cells. Other cell types may be present at up to 99.995%, i.e. umbilical blood. Advantageously solutions enriched in stem cells are used, such as a solution from which erythrocytes are eliminated, and such a solution will generally contain 0.5-3% stem cells, of course, it is possible to use solutions further enriched in stem cells. The solution, while being an aqueous solution, may contain proteinaceous components, salts, stabilizers, as is generally recognized in the art.

The term “photosensitizer” is, as well recognized in the art, a substance which absorbs light energy as a result of which the photosensitizer is activated. The activated photosensitizer can subsequently react with other compounds. This may result in the photosensitizer being modified or inactivated, but more likely the photosensitizer will return to its original state (before it was activated with light), so as to form a photocatalytic cycle in which the photosensitizer can be used again. In effect, the light energy absorbed is used for the inactivation of micro-organisms.

The term “micro-organism” is understood to mean any single-cell organism, as well as viral particles. Examples of single-cell organisms are prokarytic organisms, such as bacteria, e.g. Pseudomonas species, and eukaryotic organisms such as Chlamiydia and yeasts, e.g Candida albicans.

The term “viral particle” is understood to mean any RNA or DNA virus, single- or double-stranded and with a membrane or proteinaceous coat that may occur in a stem cell-containing liquid. Examples are HIV and hepatitis B virus.

The term “pharmaceutically acceptable counterion” is understood to be any inorganic or organic negatively charged counterion such as OH⁻, Cl⁻, acetate or citrate. It goes with-out saying that there are as many counterions X as needed to neutralise the positive charge of the actual active compound I.

According to a first embodiment at least one of R₁, R₂, R₃ and R₄ is a (C₆-C₂₀) heterocyclic aryl group comprising a nitrogen atom substituted with a group chosen from (C₁-C₂₀)alkyl, (C₂-C₂₀)alkenyl, (C₁-C₂₀)alkoxy, and (C₂-C₂₀) alkynyl, each of which may be linear or branched and each of which may be substituted with one or more groups chosen from hydroxyl, and a halogen atom. Preferably, the heterocyclic aryl group is a pyridinium group, the nitrogen of which is substituted with a (C₁-C₄) alkyl group.

According to a second embodiment at least one of R₁, R₂, R₃ and R₄ is a (C₆-C₂₀) aryl group substituted with an amino which may be substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which may be substituted with one or more groups chosen from hydroxyl, and a halogen atom. Preferably the aryl group is a trialkyl aminophenyl group where alkyl is independently (C₁-C₃) alkyl.

It has been found that quaternary nitrogen atoms in groups as defined above are very suitable for eliminating viral particles Gram-positive and Gram-negative bacteria while maintaining the integrity of the stem cells.

Preferably at least two of R₁, R₂, R₃ and R₄ comprise a quaternary nitrogen atom, and more preferably three of R₁, R₂, R₃ and R₄ comprise a quaternary nitrogen atom. Such compounds appear to give the best result.

The invention will now be elucidated with reference to the exemplary embodiments and the drawing, in which

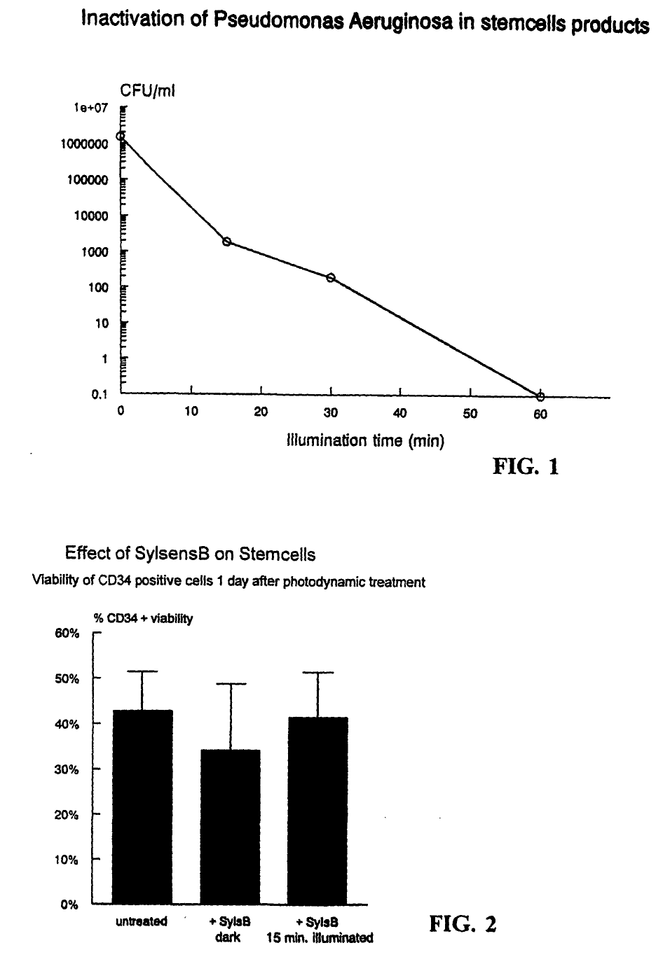

FIG. 1 shows the effective elimination of a bacterium from a stem cell-containing solution;

FIG. 2 shows the viability of stem cells before and after treatment;

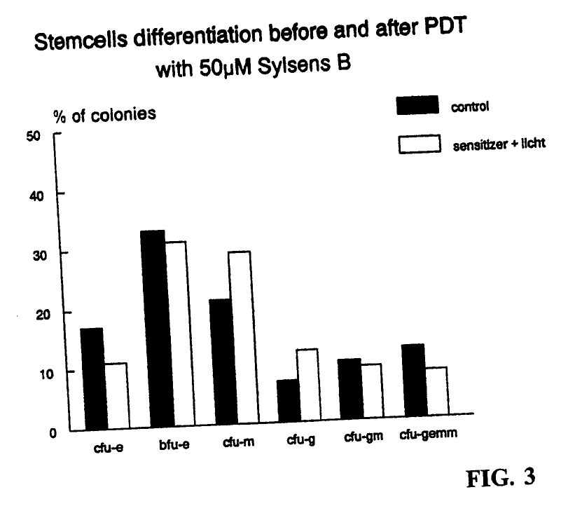

FIG. 3 represents a bar diagram to show the effect on the stem cell differentiation in comparison with untreated cells; and

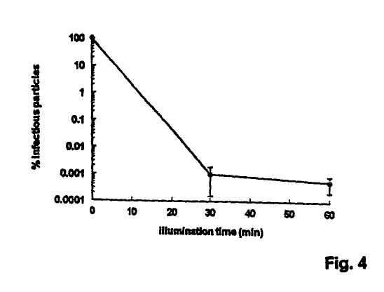

FIG. 4 shows the elimination of Vescular Stomatitus Virus (VSV)

### EXAMPLES

Stem Cells

Stem cells were isolated from fresh umbilical cord blood using hydroxy ethylstarch (HES) sedimentation (Perutelli P. et al, Vox Sang. 76(4) pp 237-40 (1999); Adorno G. et al, Clin. Lab. Haematol. 20(6) pp 341-3 (1998)). The resulting solution contained 20*10⁶ cells/ml, as determined using an automatic cell counter (Sysmex K1000, TOA Medical Electronics, Kobe, Japan).

Photodynamic Treatment of Stem Cell-Containing Solution Contaminated with Pseudomonas

The photodynamic inactivation is demonstrated using Pseudomonas aeruginosa, because of the various contaminating micro-organisms found in umbilical blood, this micro-organism was found to be the hardest to inactivate.

Stock solutions of Pseudomonas aeruginosa were added to the stem cell solution such that the volume of the spike was <10% of the total volume of the solution, and the number of bacteria was about 10⁵/ml. A sensitizer, monophenyl-tri(N-methyl-4-pyridyl)porphyrin chloride (TriP(4), Mid-Century, Posen, Ill. USA), was added to a final concentration of 25 μM. The suspension were thoroughly mixed and divided into 3-ml aliquots in polystyrene culture dishes with a diameter of 6 cm (Greiner, Alphen a/d Rijn, the Netherlands), and agitated at room temperature on a horizontal reciprocal shaker (60 cycles/min, GFL, Burgwedel, Germany) for 5 min. in the dark. The dishes (one dish per time point) were illuminated from above with a 300 W halogen lamp (Philips, Eindhoven, the Netherlands). The light passed through a 1-cm water filter, to avoid heating of the samples. A cut-off filter, only transmitting light with wavelengths above 600 nm, was used in all experiments. The irradiance at the cell layer was 35 mW/cm², as measured with an IL1400A photometer equipped with a SEL033 detector (International Light, Newburyport, Mass. USA). The following parameters were measured:

Inactivation of P. aeruginosa; and

Viability of the stem cells.

Influence of the treatment on stem cell differentiation (as measured by cfu-e, bfu-e, cfu-m, cfu-gm, and cfu-gem).

FIG. 1 shows that the bacteria were effectively killed during the treatment.

FIG. 2 shows that the viability of CD34-positive cells (stem cells) remains within the error margin for the control experiment (no illumination, no sensitizer).

FIG. 3 shows the results of the various test on stem cell differentiation (determined as described in the Mega-Cult-C Technical Manual. Assays for Quantitation of Human and Murine Megakaryocytic Progenitors. version 3.0.3, March 1999, Stem Cell Technologies Inc., Vancouver, Canada). In short, 5000 white blood cells containing the stem cells are plated on a Petri dish. The data shown in FIG. 3 are for cells from one blood sample. Table 1 shows, in addition, the data for four blood samples, and table 2 shows the averages of the five blood cell samples, confirming the result graphically depicted in FIG. 3. These results indicate that there is no significant effect of the treatment on the stem cell differentiation despite the fact that in this experiment no further measures disclosed in the state of the art to protect cells were taken.

TABLE 1

| | no of cells | | | |
|---|---|---|---|---|
| | per dish | | Control | PDT (60′) |
| st0809 | 5000 | CFU-e | 53 | 49 |
| | | CFU-GM | 5 | 6 |
| | | CFU-GEM | 16 | 8 |
| st0817 | 5000 | CFU-e | 58 | 36 |
| | | CFU-GM | 15 | 13 |
| | | CFU-GEM | 7 | 8 |
| st1026 a | 5000 | CFU-e | 39 | 11 |
| | | CFU-GM | 15 | 19 |
| | | CFU-GEM | | |
| st1026 b | 5000 | CFU-e | 50 | 44 |
| | | CFU-GM | 9 | 6 |
| | | CFU-GEM | 18 | 9 |
| st457 | 5000 | CFU-e | 33 | 34 |
| | | CFU-GM | 8 | 8 |
| | | CFU-GEM | 0,4 | 0,2 |

TABLE 2

| | control | sd | PDT (60′) | sd |
|---|---|---|---|---|
| CFU-e | 46, 6 | 10 | 34, 8 | 15 |
| CFU-GM | 10, 4 | 4 | 10, 4 | 6 |
| CFU-GEM | 10, 35 | 8 | 6, 3 | 4 |
| CFU-Mk | 37 | 1 | 38 | |

For the experiments of FIG. 2 and 3, the duration of the illumination was 1 hour.

Inactivation of VSV

5 logs of VSV were added to 1 ml of the stemcell product, which was thereafter illuminated in the presence of 50 μM Sylsens at 10 mW/cm².

After photodynamic treatment, samples were diluted 10 times in DMEM containing 2% FCS and centrifuged at 1000 rpm during 10 min. The supernatant is used for the virus assay (as described in PCT/NL99/00387).

The stem cell concentrate has been prepared as previously described for the colony assay or bacteria inactivation

5 log kill of VSV could be reached easily following this protocoll as shown in FIG. 4.

ABBREVIATIONS

| | |
|---|---|
| CFU-E | Colony-forming unit-erythroid produces 8-200 eryth- |
| | roblasts in 1-2 clusters. Each cluster must contain |
| | a minimum of 8 erythroblasts to be scored refer to |
| | progenitors that give rise to the smallest and most |
| | rapidly maturing erythroid colonies. |
| BFU-E | Burst-forming-unit erythroid produces 3 of more |
| | small clusters or one large cluster containing more |
| | than 200 erythroblasts and extremely large but pure |
| | erythroblast colonies containing 16 of more and |
| | 10.000 of more individual cells. |
| | It is the class of more primitive erythroid progeni- |
| | tors than CFU-E, have a greater proliferative capac- |
| | ity which enables it to give rise to larger, multi- |
| | clustered erythroid colonies than those produced |
| | from CFU-E. |
| | Mature EF are immediate precursors of CFU-E, con- |
| | tains between 3 and 8 clusters. |
| | Primitive BFU-E are those progenitors that give rise |
| | to 9 or more clusters of erythroblasts. Because of |
| | difficulties in distinguishing CFU-E and BFU-E, they |
| | have been taken together here and designated CFU-E. |
| CFU-G | Clonogenic progenitors of granulocytes containing |
| | more than 20 cells. |
| CFU-M | Clonogenic progenitors of macrophages, containing |
| | more than 20 cells macrophages have a concentrated |
| | central core, can become very large. |
| CFU-GM | Colony-forming unit-granulocyte-macrophase produces |
| | 20 or more granulocytes and macrophages. |
| CFU-GEM | Colony-forming unit-granulocyte, erythroid, macro- |
| | phage megakaryocyte produces 20 or mroe cells. |
| | Such colonies are best evaluated after a minimum of |
| | 18 days of growth in media containing leukocyte con- |
| | ditioned media, or after 14 days to 16 days when re- |
| | combinant growth factors are used. In the latter |
| | case, extra care must be taken when scoring multi- |
| | lineage colonies. In some small numbers of granulo- |
| | cytes, macrophages and/or megakaryocyten may appear |
| | around the periphery of a sperical mass of hemoglo- |
| | binized erythroid cells. Multi-lineage colonies of |
| | this type can be mistakenly scored as pure erythroid |
| | colonies if not examined under high power. |

1. Method of inactivating micro-organisms present in a liquid containing stem cells comprising the steps of
combining said liquid with a photosensitiser, and
activating the photosensitiser,
characterized, in that as the photosensitiser a compound is used chosen from the group consisting of compounds with the formulas Ia-Id 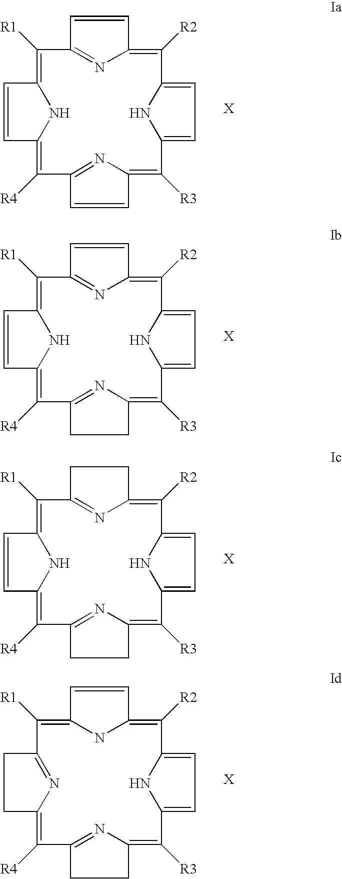
wherein R₁, R₂, R₃ and R₄ are independently chosen from the group consisting of
hydrogen,
a halogen atom,
(C₁-C₂₀)alkyl, (C₁-C₂₀)alkoxy, (C₁-C₂₀)acyl, (C₁-C₂₀)acyloxy, (C₂-C₂₀)alkenyl, or (C₂-C₂₀)alkynyl, each of which may be linear or branched and each of which may be substituted with one or more groups chosen from
hydroxyl,
amino which may be substituted with 1 to 3 groups chosen from (C₁-C₂₀)alkyl, (C₂-C₂₀)alkenyl, (C₁-C₂₀)alkoxy, (C₂-C₂₀)alkynyl, and -(R₅-Z)ₘ-R₆ where R₅ is (CH₂)ₙ, Z is O or S, and R₆ is (C₁-C₂₀)alkyl and m and n are, independently, 1-10, each substituent group of the amino group may be linear or branched and each of these may be substituted with one or more groups chosen from hydroxyl, and a halogen atom,
nitril, and
a halogen atom,
(C₆-C₂₀)aryl, and (C₆-C₂₀) heterocyclic aryl group each of which may be substituted with one or more groups chosen from
hydroxyl,
amino which may be substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which may be substituted with one or more groups chosen from hydroxyl, and a halogen atom,
nitril,
a halogen atom, and
(C₁-C₁₀)alkyl, (C₁-C₁₀)alkoxy, (C₂-C₁₀)alkenyl, the heterocyclic aryl group containing at least one atom chosen from N, O, P, and S where P, N or S may be substituted with a group chosen from (C₁-C₂₀)alkyl, (C₂-C₂₀)alkenyl, (C₁-C₂₀)alkoxy, and (C₂-C₂₀)alkynyl, each of which may be linear or branched and each of which may be substituted with one or more groups chosen from hydroxyl, and a halogen atom,
at least one of the groups R₁, R₂, R₃ and R₄ contains a quaternary nitrogen atom, and wherein X is a pharmaceutically acceptable counterion.

2. Method according to claim 1, characterized, in that at least one of R₁, R₂, R₃ and R₄ is a (C₆-C₂₀) heterocyclic aryl group comprising a nitrogen atom substituted with a group chosen from (C₁-C₂₀)alkyl, (C₂-C₂₀)alkenyl, (C₁-C₂₀)alkoxy, and (C₂-C₂₀)alkynyl, each of which may be linear or branched and each of which may be substituted with one or more groups chosen from hydroxyl, and a halogen atom.

3. Method according to claim 2, characterized, in that the heterocyclic aryl group is a pyridinium group, the nitrogen of which is substituted with a (C₁-C₄) alkyl group.

4. Method according to claim 1, characterized in that at least one of R₁, R₂, R₃ and R₄ is a (C₆-C₂₀) aryl group substituted with an amino which may be substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which may be substituted with one or more groups chosen from hydroxyl, and a halogen atom.

5. Method according to claim 4, characterized in that the aryl group is a trialkyl aminophenyl group where alkyl is independently (C₁-C₃) alkyl.

6. Method according to any of the preceding claims, characterized in that at least two of R₁, R₂, R₃ and R₄ comprise a quaternary nitrogen atom.

7. Method according to claim 6, characterized in that three of R₁, R₂, R₃ and R₄ comprise a quaternary nitrogen atom.
